(19) United States
(12) Patent Application Publication
(10) Pub. No.: US 2024/0281486 A1
(43) Pub. Date: Aug. 22, 2024

(54) HEALTHCARE PROVIDER RANKING

(71) Applicant: Electronic Referral Manager Inc., Mt. Pleasant, SC (US)

(72) Inventors: Richard Hammer, Tucson, AZ (US); Ivan Nolasco, Santo Domingo (DO); David Bongiovanni, Mt. Pleasant, SC (US)

(73) Assignee: Electronic Referral Manager Inc., Mt. Pleasant, SC (US)

(21) Appl. No.: 18/110,674

(22) Filed: Feb. 16, 2023

(51) Int. Cl. G06F 16/9538; G06F 16/951; G16H 40/20

(52) U.S. Cl. CPC G06F 16/9538; G06F 16/951; G16H 40/20

(57) ABSTRACT

A method for ranking search results includes creating a search engine index, importing healthcare provider data into the search engine index, the healthcare provider data including individual providers and healthcare organizations, indexing the healthcare provider data, calculating a provider score for each healthcare provider, uploading the provider score to the search engine index, receiving a search request from a user, and outputting a result of the search request to the user on a display, wherein the result of the search request is shown with an associated provider score.

### BACKGROUND

The present invention relates to managing healthcare referrals, and more specifically, to searching for healthcare providers and providing the results of the search in a useful manner to users.

In the medical industry, patients are often recommended or referred to a healthcare provider by another healthcare provider. These referrals are often made by, for example, a primary care provider to a specialist provider. For example, if a patient is experiencing a skin condition, the primary care provider may refer the patient to a dermatologist for more specialized care. The referral results in an appointment for the patient with a specialist—a dermatologist in this example.

### SUMMARY

Embodiments of the present invention are directed to a method for ranking search results includes creating a search engine index, importing healthcare provider data into the search engine index, the healthcare provider data including individual providers and healthcare organizations, indexing the healthcare provider data, calculating a provider score for each healthcare provider, uploading the provider score to the search engine index, receiving a search request from a user, and outputting a result of the search request to the user on a display, wherein the result of the search request is shown with an associated provider score.

Embodiments of the present invention are directed to a system for ranking search results the system is operative to create a search engine index, import healthcare provider data into the search engine index, the healthcare provider data including individual providers and healthcare organizations, index the healthcare provider data, calculate a provider score for each healthcare provider, upload the provider score to the search engine index, receive a search request from a user, and output a result of the search request to the user on a display, wherein the result of the search request is shown with an associated provider score.

Embodiments of the present invention are directed to a non-transitory computer readable medium including instructions for creating a search engine index, importing healthcare provider data into the search engine index, the healthcare provider data including individual providers and healthcare organizations, indexing the healthcare provider data, calculating a provider score for each healthcare provider, uploading the provider score to the search engine index, receiving a search request from a user, and outputting a result of the search request to the user on a display, wherein the result of the search request is shown with an associated provider score.

### BRIEF DESCRIPTION OF THE DRAWINGS

FIG. 1 illustrates a block diagram of an example computer system.

FIG. 2 illustrates a block diagram showing how healthcare provider referral data is organized in a database and scored in a search engine index.

FIG. 3 illustrates a block diagram describing how the search engine index is created.

FIG. 4 illustrates a block diagram of a method for importing provider data into the search engine index.

FIG. 5 illustrates a block diagram of a method of indexing provider and organization details.

FIG. 6 illustrates a block diagram of a method for calculating provider scores for each provider.

FIG. 7 illustrates a block diagram of a method that provides search results to a user.

### DETAILED DESCRIPTION

Although this disclosure includes a detailed description of a computing environment, the teachings herein are not limited to the described computing environment. In embodiments, any implementation of a computer environment may be used whether now understood or later developed.

The referral system described herein provides a system that ranks results of a search for a list of healthcare providers using a score assigned to each healthcare provider. The score resulting in a ranked list of healthcare providers is based on several factors including, for example, the geographic location of the healthcare provider, and the type of insurance accepted by the healthcare provider, among other things.

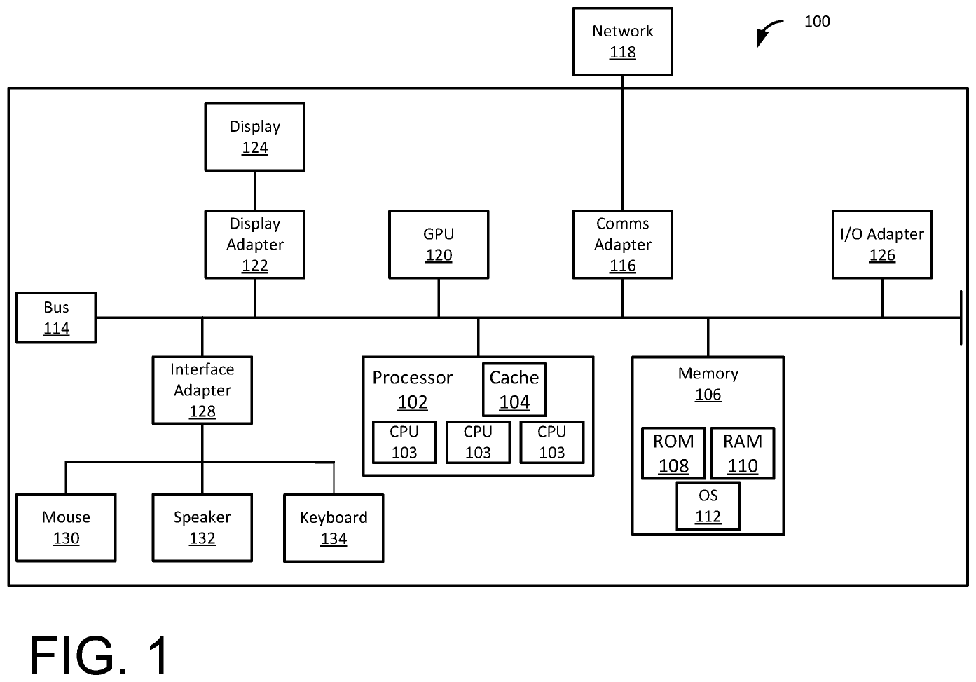

FIG. 1 illustrates a block diagram of an example computer system 100. In embodiments, a microprocessor is arranged in a personal computer, workstation, minicomputer, or mainframe computer. Embodiments system 100 a mobile device or part of a mobile device.

The system (computer) 100 includes processor 102 communicatively connected to an input/output (I/O) adapter 126, memory 106, a communications adapter 116, an interface adapter 128, a display adapter 122, and a graphics processing unit (GPU) 120 via a system bus 114.

The processor 102 includes any number of central processing units (CPU) 103 and a cache memory 104. The CPU 102 is operable to perform any number of processing tasks by executing program instructions. In embodiments the processor 102 may include any number of suitable auxiliary processors or microprocessors. The processor 102 may include a cache memory 104 that is often operative to increase the speed of processing tasks performed by the processor 102.

The I/O adapter 126 connects a variety of input and output devices to the computer 100. The memory 106 may include any type of suitable memory such as, for example, magnetic, solid state, disk-based storage, drum-based storage, random access memory (RAM) 110, and read only memory (ROM) 108. The operating system of the system 100 may be stored in the memory 106. The communications adapter 116 is operative to facilitate communications between the system 100 and a network 118. The network 118 may include, for example, a local area network, a wide area network or the Internet. The system 100 may connect to the network 118 via, for example, a wired or wireless connection. The display adapter 122 is operative to communicably connect a display 124 to the system 100. The display may include any type of suitable display such as, for example, a desktop display, a touch screen, or a mobile display. The GPU 120 includes a processor that is operative to generate graphical data that may be sent to the display 124 for presentation to a user. The interface adapter 128 provides an interface between peripheral devices and the system bus 114. The peripheral devices may include, for example, a mouse 130, a speaker 132, and a keyboard 134, sensors, and actuators, among others.

The memory 106 may store computer-readable and computer-executable instructions. The instructions implement logical functions. The operating system 112 may control the operations of the system 100 and the functions of other software programs or operations.

Memory can also include program instructions for Engine 1, configured to improve the wellness of a user.

According to some embodiments, computer 100 can include a mobile communications adapter 123. Mobile communications adapter 123 can include GPS, cellular, mobile, and/or other communications protocols for wireless communication.

In some embodiments, computer 100 can further include communications adapter 116 for coupling to a network 118.

As discussed above, the healthcare referral process often involves a first healthcare provider providing a referral to a second healthcare provider. The second healthcare provider provides additional services to the patient that may not be available from the first healthcare provider.

It is challenging to find the correct healthcare provider for patient referrals. Indeed, many factors may be considered when referring a patient to another healthcare provider. For example, the type of medicine practiced by the referred healthcare provider may be a factor. However, other factors may also be relevant such as, for example, the geographic location of the second healthcare provider, the gender of the provider, the type of insurance accepted by the provider, whether the provider accepts new patients, and wait times for patient appointments, among others.

In a referral system, healthcare providers are input into the system from a variety of sources including, for example, external databases and proprietary databases. These sources are combined into a primary database that may include data from, for example, the Centers for Medicare and Medicaid Services (CMS) referral data, and CMS National Provider Identifier (NPI) data.

The use of such databases may be used to match a patient with a provider however, such matching may be difficult if the matching is not tailored to the needs of the patient. For example, a long list of possible referred healthcare providers may not be as helpful to the user as a list of potential providers that is ranked based on a number of criteria that best match the referral provider to the needs of a user or patient.

The system 100 includes referral data that includes NPI provider data (healthcare provider data) and NPI organization data (organizations such as, for example, associated medical practices, hospitals, etc.). The referral data may include any number of types of data. For example, the referral data may include a healthcare provider (provider) database that may be retrieved from the NPI data. The healthcare provider database includes the name of a provider, the specialty of a provider, a hospital associated with the provider, who the provider sends referrals to, and who the provider receives referrals from. Other data includes whether the provider will accept new patients, the insurance networks accepted by the provider, the specialties of the provider, common procedures performed by the provider, demographic information, average wait and response times, the language spoken by the provider, the gender of the healthcare provider, International Classification of Diseases, 10th revision (ICD-10), and the geographic location of the provider. The IDC-10 is a classification and coding system for medical diagnoses and procedures managed by the World Health Organization. The healthcare provider database may also include, for example, average wait and response times, healthcare provider ratings and reviews, fees for services, associations with groups, education, specialties, titles, and credentials. Other categories that may be included include Current Procedural Terminology (CPT) codes which is a classification and coding system for medical procedures and services managed by the American Medical Association. Taxonomy codes may be included; taxonomy codes are a classification and coding system for healthcare provider specialties managed by the National Uniform Claim Committee (NUCC). Other codes may include, for example, Systemized Nomenclature of Medicine Clinical Terms classification and coding system for medical terms. This may include conditions and procedures and is managed by the International Health Terminology Standards Development Organization (IHTSDO).

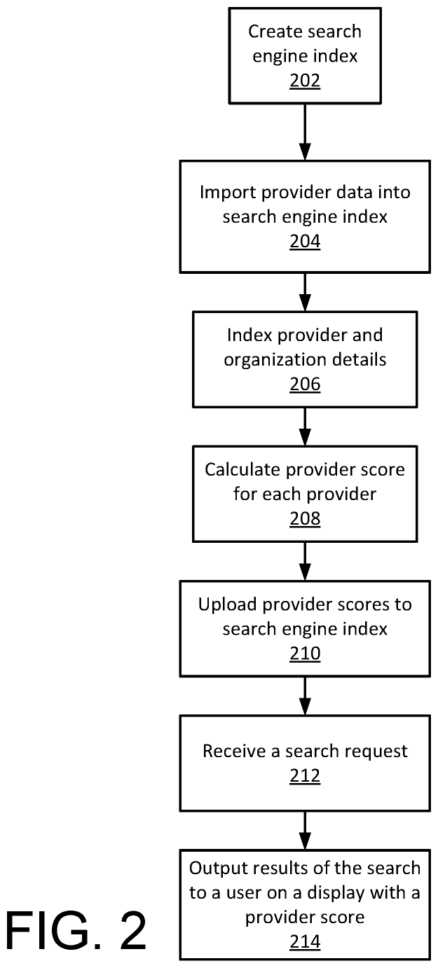

In this regard, FIG. 2 illustrates a block diagram showing how healthcare provider referral data is organized in a database and scored in a search engine index. The system is operative to perform a search for one or more healthcare providers and assign a score to one or more providers. The scores are then used to rank the providers and present the ranked providers to a user on the screen 124 (of FIG. 2).

In block 202, a search engine index is created. The search engine index is optimized for searching and allows the system to define and index data that is associated with providers and organizations. In block 204, provider data is imported into the search engine index. Provider data is data that is associated with a healthcare provider.

Provider data includes, for example, the name of the healthcare provider, the gender, healthcare specialties and affiliated organizations. Organization data is data associated with an organization, hospital, or healthcare provider practice. Organization data includes the name, insurance accepted, and other details associated with an organization. In block 206, provider and organization details are indexed. The provider and organization details are indexed by fetching the provider and organization data from the database. The database fields for each record are normalized into corresponding data types defined in the index mappings, and the normalized database record data is uploaded to the search index.

A provider score is calculated for each provider in block 208. A provider score is a rating such as, for example, a numerical score from 0 to 100 that is calculated based on criteria. Each criterion may be weighed individually. Some criteria may be static in that they are computed while loading provider data. For example, whether a provider is affiliated with an organization may be a static criterion. Other criteria may be loaded at runtime depending on a reference point. For example, proximity, which is based on the geospatial coordinates of a specific patient location with respect to the location of a healthcare provider. As discussed above, criteria may be associated with a weighting. Some examples of score weights may include: appointment wait time a—10 points, healthcare provider rating—20 points, network tier—30 points, insurance accepted by the healthcare provider—30 points, and proximity of the healthcare provider to the location of the patient—10 points. Similar score weights may be used to rank an organization.

A healthcare provider rating may be, for example, a number in a range of numbers such as 1 through 5 that represents a qualitive value assigned by users in the system to a healthcare provider. A higher rating indicates a better score.

Network tiers may be assigned to healthcare providers within a specific network as defined by an organization. For example, a first tier may be assigned to employed providers, a second tier may be assigned to contract providers, and a third tier may be assigned to independent providers. A network tier describes a professional relationship between a healthcare organization and a healthcare provider. This includes, for example, an employment relationship, a position as an independent contractor, or granting privileges by the organization. The affiliation may have several levels such as, for example, a first level “1” where 1 represents a close affiliation of a healthcare provider and a healthcare organization and a higher number represents a looser affiliation between a healthcare provider and a healthcare organization. A network tier may be used in the calculation of a provider score such as, for example, a number of points that may be customized per account for example, 30 points, or a percentage may be customized per account. The network tier score for a given provider may be calculated using a function where the network tiers are assigned point values. For example, a tier 1 may be assigned 100% of the points, a tier 2 may be assigned 50% of the points, and a tier 3 may be assigned 30% of the points.

The appointment wait time is the average number of days between when a referral is received to the appointment date calculated for each provider from the referral data associated with the provider. The appointment wait time score is calculated using a function where the appointment wait times are assigned point values wherein a higher wait time may be assigned fewer points, point values, or a lower percentage. In that regard, a lower wait time may be assigned more points, point values, or a higher percentage.

In block 210, the provider scores are uploaded to the search engine index. In block 208 a search request is received by the system 100 (of FIG. 1). In block 208, the results of the search request are displayed to a user on the display 124. A search request is received in block 212. Results of the search are output to a user on the display 124 in block 214.

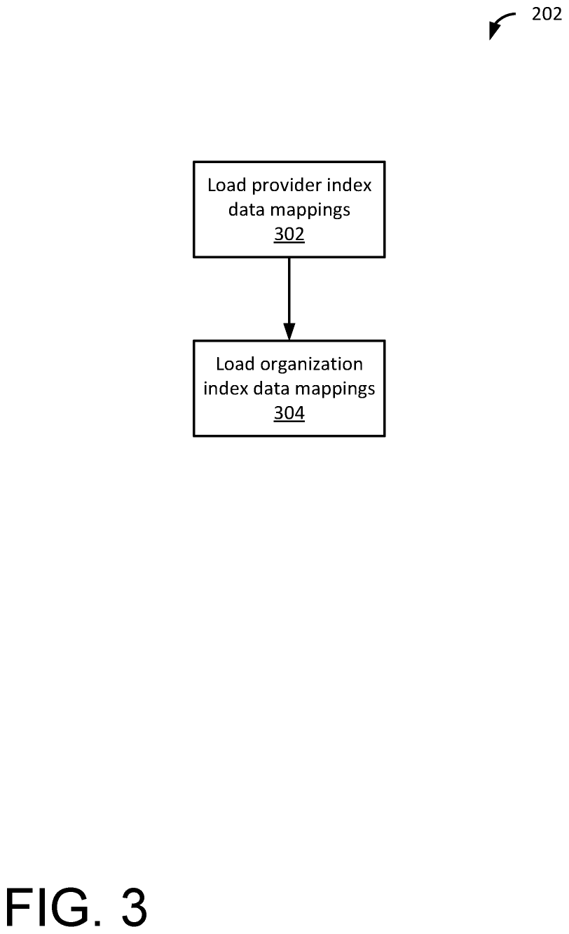

FIG. 3 illustrates a block diagram describing how the search engine index is created in block 202 (of FIG. 2). In this regard, the provider index by data mappings are loaded in block 302 (of FIG. 3). Organization index data mappings are loaded in block 304. The search engine defines how data will be indexed by defining fields of the index. Provider and organization index mappings are substantially similar though some types of data that is associated with a person may be different from data associated with an organization. Index mapping defines the data fields to keep track of the type of data in the field such as, for example, text Booleans or numbers. The index maintains a reference to the database so once a search is performed solely within the index, the corresponding full record can be pulled from the database. Each mapped field contains a line of data about the provider or organization records. For example, first name, last name, gender, longitude, latitude, rating, or specialty. For text fields the text is token into words or searchable tokens to allow the ability to conduct partial searches within the text.

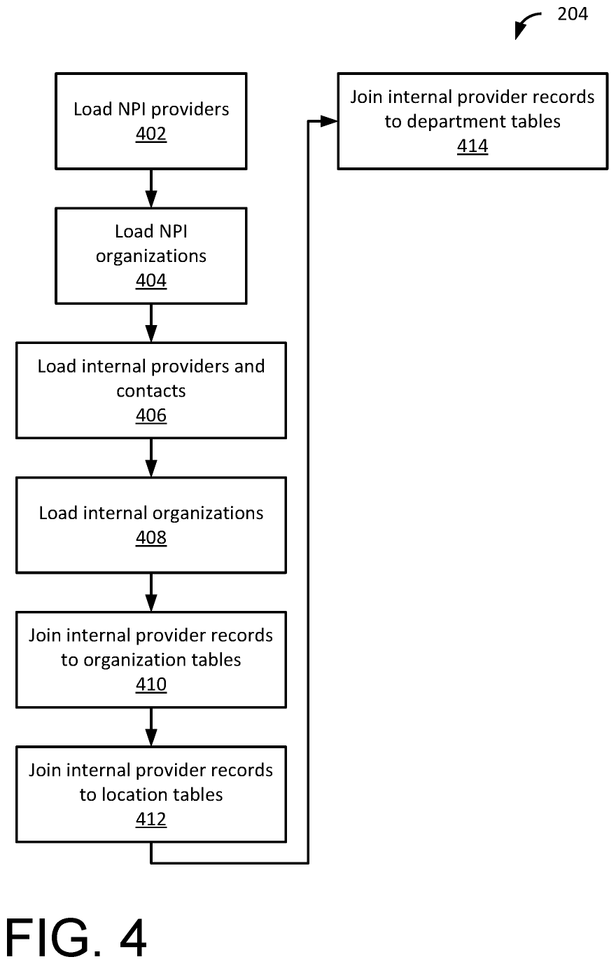

FIG. 4 illustrates a block diagram of a method for importing provider data into the search engine index as described in block 204 (of FIG. 2). In block 402 (of FIG. 4) NPI providers are loaded into the database tables. In block 404, NPI organizations are loaded into the database tables. Other provider records (an internal list of providers from a list of providers) and contact records (an internal or proprietary list of contacts from a list of contacts) are loaded into database tables in block 406. In block 408, other organizations (an internal or proprietary list of organizations from a list of organizations) are loaded into database tables. In block 410 internal provider records are joined to organization tables. The internal provider records may be joined to the organization tables by, for example, a link between the internal provider records and the organization tables.

In block 412 internal provider records are joined to location tables. Location tables contain data of each of the locations that are assigned to the organization or provider. They contain, for example, the names, phone numbers, addresses, and geolocation coordinates of the location. If an organization includes multiple locations a healthcare provider may be assigned to multiple locations.

In block 414 internal provider records are joined to department tables. Department tables contain each of the departments or specialties within a specific location. The tables contain the name and specialties of the department, and a reference to the location and parent organization. A healthcare provider may be assigned to a location indirectly through a department. In this regard, the internal provider records are joined to location tables by, for example, reading one record at a time, normalizing the data into data types optimized for searching such as, for example, text strings and numbers, and batching the records to allow large data sets to be processed without substantially taxing computational resources such as, for example, processing time and memory resources.

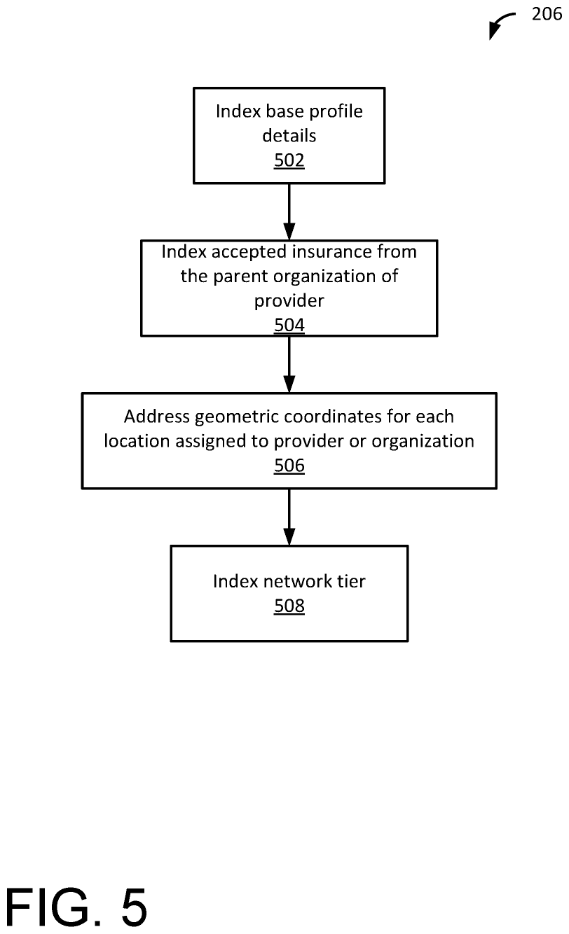

FIG. 5 illustrates a block diagram of a method of indexing provider and organization details as described in block 206 (of FIG. 2). Referring to FIG. 5, in block 502 base profiled details are indexed. A base profile is data associated with a provider that is indexed such as, for example, name or gender as opposed to data associated with the operations of the provider such as, for example, referral wait time that depends on the referral data (and is recalculated when the referral data changes), or accepted insurance that often depends on the insurance accepted by the organization.

In block 504, accepted insurance from the parent organization of a provider and, in some cases, the provider are indexed. A parent organization may include, for example, a company that is affiliated with providers where the company serves an administrative roll for the providers. In block 506, geographic coordinates for each provider are indexed and assigned to each provider or organization. In block 508, an network tier is indexed. An network tier describes whether a provider is affiliated with an organization. For example, whether a provider is affiliated with an organization or network.

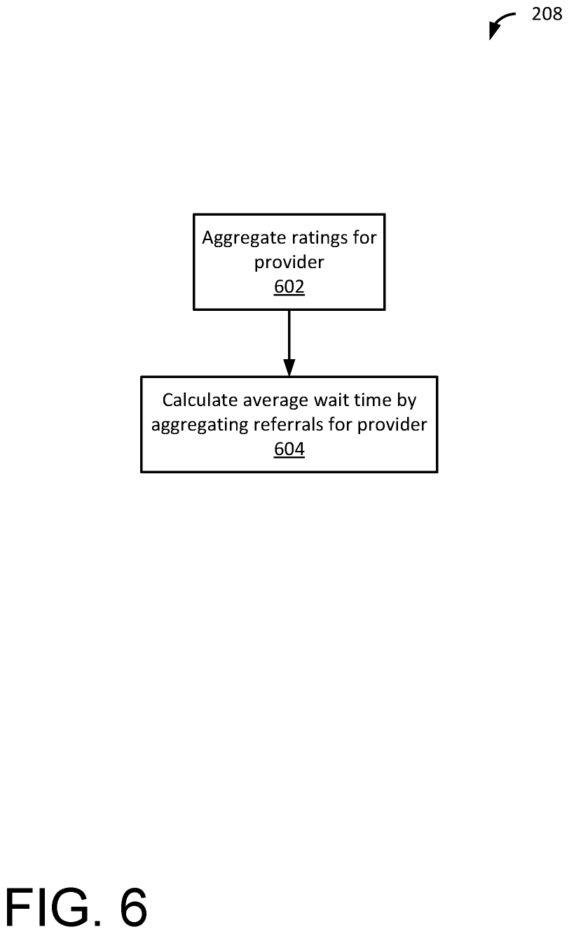

FIG. 6 illustrates a block diagram of a method for calculating provider scores for each provider as described in block 208 (of FIG. 2). Referring to FIG. 6, ratings are aggregated for each provider in block 602. In block 604, an average wait time for a provider is calculated by aggregating the referrals for the provider. The provider scores may be assigned by a user or users while accessing a provider directory or a provider details page. For example, a user may provide a rating from 1 to 5 to a provider. The system may aggregate these user ratings to calculate, for example, an average score of the provider rankings.

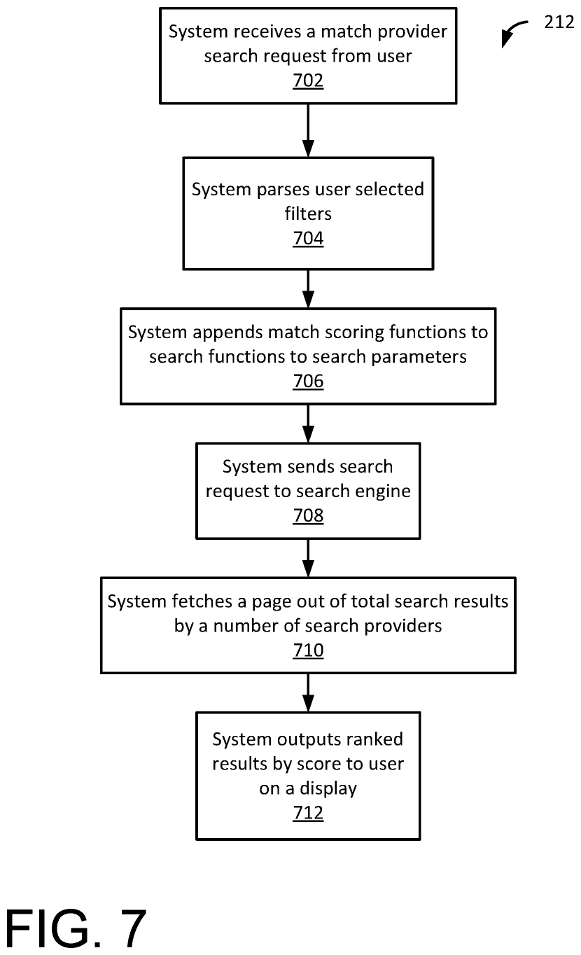

FIG. 7 illustrates a block diagram of a method that provides search results to a user. Following the uploading the provider scores to the search engine index, search results may be provided to a user. In this regard, in block 702, the system receives a match provider search request from a user. For example, a user inputs a request for a provider. In block 704, the system parses filters selected by the user. The filters may include, for example, a specialty of a provider, a geographic location of a provider, whether the provider is in a particular insurance network, and favorite providers of the user. There may be other filters including, for example, substantially any mapped filed in the index may be used as a filter.

In block 706, the system 100 (of FIG. 1) appends match scoring functions to the search parameters based on the account settings of the user. For example, a provider rating function distributes the total score points applicable to it using criteria such as, for example, five stars—100% of total score points, four stars—75% of total score points, three stars—50% of total score points, two stars—25% of total score points, one star-5% of total score points. An account may define how many points out of the total 100 to assign to this function for example, having four and five stars each award 100% of the total score points.

In block 708 (of FIG. 7), the system 100 sends the search request to an Elasticsearch engine, which is a search engine that provides a distributed, multitenant-capable full-text search engine with an HTTP web interface. In this regard, the request adds query filters for each selected filter to limit the results of the search. The request appends the scoring functions to qualify the score of each matching record. The elastic search finds the providers that match the selected filters.

In block 710, the system 100 (of FIG. 1) fetches a page out of the total search results by a number of search providers. In block 712, the system outputs the results of the search to a user on the display 124 (of FIG. 1).

The referral system described herein provides a system that effectively ranks results of a search for potentially referred healthcare providers. The ranked list displays providers based on several factors including, for example, the practice area of a healthcare provider, the gender of the healthcare provider, the geographic location of the healthcare provider, and the type of insurance accepted by the healthcare provider, among other things. For example, the list of healthcare providers may be sorted or ranked based on the score of each healthcare provider. In another example, one or more healthcare providers may be displayed with their associated scores.

The present invention may include a computer program, a method, system, and/or a computer program product in any level of integration. A computer program product can comprise of any type of computer readable storage medium that includes computer readable program instructions that causes a processor to implement some or all aspects of the invention.

The computer readable storage medium may include any suitable device that may store instructions for use by a computer, processor or other similar device. The computer readable storage medium may include, for example a random access memory, a read only memory, a flash memory, a magnetic or light readable disk. A computer readable storage medium as used herein does not include transitory signals such as, for example, electromagnetic waves.

The computer readable program instructions may be received by a computer or processing device by any suitable process such as, for example, wireless, wired, or optical transmission via a network or other communications devices or processes.

Each block of the illustrations and block diagrams described herein can be implemented by computer readable program instructions, and input to a processor of a computer to carry out or execute the computer readable program instructions.

The descriptions of the various embodiments are disclosed for illustration, but are not intended to limit the disclosed embodiments. Many modifications and variations will be apparent to those of ordinary skill in the art without departing from the scope of the described embodiments.

What is claimed is:

1. A method for ranking search results, the method comprising:
creating a search engine index;
importing healthcare provider data into the search engine index, the healthcare provider data including individual providers and healthcare organizations;
indexing the healthcare provider data;
calculating a provider score for each healthcare provider;
uploading the provider score to the search engine index;
receiving a search request from a user; and
outputting a result of the search request to the user on a display, wherein the result of the search request is shown with an associated provider score.

2. The method of claim 1, wherein the creating the search engine index includes:
loading healthcare provider index data mappings; and
loading healthcare organization index data mappings into the search engine index.

3. The method of claim 1, wherein the importing healthcare provider data into the search engine index includes:
loading National Provider Identifier (NPI) data into the search engine index;
loading internal provider records and contacts data into the search engine index;
loading internal organizations into the search engine index;
joining the internal provider records to organization tables;
joining the internal provider records to location tables; and
joining the internal provider records to department tables.

4. The method of claim 1, wherein the indexing healthcare provider data and healthcare provider organization data includes:
fetching the healthcare provider and organization data from a database;
normalizing database fields for each record into corresponding data types defined in index mappings; and
uploading normalized database record data into the search engine index.

5. The method of claim 1, wherein the calculating a provider score for each healthcare provider includes:
aggregating ratings for each provider; and
calculating an average wait time by aggregating referrals for the providers.

6. The method of claim 1, wherein the receiving a search request from the user includes:
receiving a match provider search request from the user;
parsing user selected filters;
appending match scoring functions to search functions to search parameters;
sending the search request to a search engine;
fetching a page out of total search results by a number of search providers; and
outputting the results of the search to the user on the display, wherein the search providers are displayed ranked as a function of the provider score associated with each provider.

7. The method of claim 6, wherein the matching scoring functions to search functions to search parameters includes:
retrieving an account setting from an account;
applying the account setting as an input to a corresponding matching function;
applying specific search parameters from a request as input to the corresponding matching function;
running the corresponding matching function against a record in an index; and
computing a score for the record.

8. A system for ranking search results operative to:
create a search engine index;
import healthcare provider data into the search engine index, the healthcare provider data including individual providers and healthcare organizations;
index the healthcare provider data;
calculate a provider score for each healthcare provider;
upload the provider score to the search engine index;
receive a search request from a user; and
output a result of the search request to the user on a display, wherein the result of the search request is shown with an associated provider score.

9. The system of claim 8, wherein the creating the search engine index includes:
loading healthcare provider index data mappings; and
loading healthcare organization index data mappings into the search engine index.

10. The system of claim 8, wherein the importing healthcare provider data into the search engine index includes:
loading National Provider Identifier (NPI) data into the search engine index;
loading internal provider records and contacts data into the search engine index;
loading internal organizations into the search engine index;
joining the internal provider records to organization tables;
joining the internal provider records to location tables; and
joining the internal provider records to the department tables.

11. The system of claim 8, wherein the indexing healthcare provider data and healthcare provider organization data includes:
fetching the healthcare provider and organization data from a database;
normalizing database fields for each record into corresponding data types defined in index mappings; and
uploading normalized database record data into the search engine index.

12. The system of claim 8, wherein the calculating a provider score for each healthcare provider includes:
aggregating ratings for each provider; and
calculating an average wait time by aggregating referrals for the providers.

13. The system of claim 8, wherein the receiving a search request from the user includes:
receiving a match provider search request from the user;
parsing user selected filters;
appending match scoring functions to search functions to search parameters;
sending the search request to a search engine;
fetching a page out of total search results by a number of search providers; and
outputting the results of the search to the user on the display, wherein the search providers are displayed ranked as a function of the provider score associated with each provider.

14. The system of claim 13, wherein the matching scoring functions to search functions to search parameters includes:
retrieving an account setting from an account;
applying the account setting as an input to a corresponding matching function;
applying specific search parameters from a request as input to the corresponding matching function;
running the corresponding matching function against a record in an index; and
computing a score for the record.

15. A non-transitory computer readable medium including instructions for:
creating a search engine index;
importing healthcare provider data into the search engine index, the healthcare provider data including individual providers and healthcare organizations;
indexing the healthcare provider data;
calculating a provider score for each healthcare provider;
uploading the provider score to the search engine index;
receiving a search request from a user; and
outputting a result of the search request to the user on a display, wherein the result of the search request is shown with an associated provider score.

16. The non-transitory computer readable medium of claim 15, wherein the creating the search engine index includes:
loading healthcare provider index data mappings; and
loading healthcare organization index data mappings into the search engine index.

17. The non-transitory computer readable medium of claim 15, wherein the importing healthcare provider data into the search engine index includes:
loading National Provider Identifier (NPI) data into the search engine index;
loading internal provider records and contacts data into the search engine index;
loading internal organizations into the search engine index;
joining the internal provider records to organization tables;
joining the internal provider records to location tables; and
joining the internal provider records to the department tables.

18. The non-transitory computer readable medium of claim 15, wherein the indexing healthcare provider data and healthcare provider organization data includes:
fetching the healthcare provider and organization data from a database;
normalizing database fields for each record into corresponding data types defined in index mappings; and
uploading normalized database record data into the search engine index.

19. The non-transitory computer readable medium of claim 15, wherein the calculating a provider score for each healthcare provider includes:
aggregating ratings for each provider; and
calculating an average wait time by aggregating referrals for the providers.

20. The non-transitory computer readable medium of claim 15, wherein the receiving a search request from the user includes:
receiving a match provider search request from the user;
parsing user selected filters;
appending match scoring functions to search functions to search parameters;
sending the search request to a search engine;
fetching a page out of total search results by a number of search providers; and
outputting the results of the search to the user on the display, wherein the search providers are displayed ranked as a function of the provider score associated with each provider.
